(19) United States
(12) Patent Application Publication
(10) Pub. No.: US 2004/0078843 A1
(43) Pub. Date: Apr. 22, 2004

(54) Method for controlling harmful organisms in the culture of useful plants

(76) Inventor: Manfred Kern, Traminerweg Frankfurt (DE)

Correspondence Address: BURNS DOANE SWECKER & MATHIS L L P, POST OFFICE BOX 1404, ALEXANDRIA, VA, 22313-1404, US

(21) Appl. No.: 10/470,804

(22) PCT Filed: Jan. 17, 2002
(86) PCT No.: PCT/EP02/00423

(30) Foreign Application Priority Data
Feb. 3, 2001 (DE) 101-04-871.8

(51) Int. Cl. A01N 65/00; A01N 57/00; A01H 1/00; C12N 15/82; A01N 47/10

(52) U.S. Cl. 800/279; 514/67; 514/120; 514/479

(57) ABSTRACT

Method for controlling harmful organisms in genetically modified vegetable plants containing a gene derived from Bacillus thuringiensis, said gene encoding and expressing a protein with an insecticidal action, wherein an insecticidally active quantity of one or more compounds from the following groups (a) to (f) is applied to the plants, their seeds or propagation material and/or the area in which they are cultivated:

a) insecticidal organophosphorus compounds;

b) pyrethroids;

c) insecticidal carbamates;

d) biopesticides;

e) insecticidal growth regulators;

f) others.

The inventive method makes possible a reduced application rate of crop protectants which act synergistically with the transgenic plants, in addition to an increased and wider-ranging efficiency of said transgenic plants, thus offering economic and ecological advantages.

The invention relates to a method for controlling harmful organisms in crops of Bt vegetables.

Genetically modified vegetable plants such as aubergines which express Bacillus thuringiensis (Bt) toxins and are therefore resistant to attack by specific harmful insects (Bt vegetables) are known and employed increasingly in commercial crop production (see, for example, U.S. Pat. No. 6,072,105). Although existing genetically modified vegetables have very good characteristics, a series of problems remains so that there is much room for improvement.

It was therefore a further object to provide as effective and environmentally friendly solutions as possible to problems in the control of vegetable pests.

Surprisingly, it has now been found that certain classes of insecticides act synergistically when used in combination with Bt vegetables.

The invention therefore relates to a method for controlling harmful organisms in genetically modified vegetable plants containing a gene derived from Bacillus thuringiensis, said gene encoding and expressing a protein with an insecticidal action, wherein an insecticidally active quantity of one or more compounds from the following groups (a) to (f) is applied to the plants, their seeds or propagation material and/or the area in which they are cultivated:

a) insecticidal organophosphorus compounds selected from the group consisting of:

acephate (4), azinphos-ethyl (40), azinphos-methyl (41), cadusafos (101), chlorfenvinphos (124), chlormephos (128), chlorpyrifos (137), demeton-S-methyl (205), diazinon (209), dicrotophos (223), dimethoate (243), disulfoton (257), ethion (283), ethoprophos (286), etrimfos (295), fonofos (366), isazofos (429), isofenphos (430), malathion (448), methamidophos (479), methidathion (481), mevinphos (499), monocrotophos (502), omethoate (533), parathion (551), phenthoate (565), phorate (570), phosalone (571), phosmet (572), phosphamidon (573), phoxim (575), pirimiphos-methyl (585), profenofos (594), prothiofos (614), pyridaphenthion (625), quinalphos (634), terbufos (690), tetrachlorvinphos (694) and triazophos (726);

b) pyrethroids selected from the group consisting of:

acrinathrin (9), allethrin (17), bifenthrin (70), cycloprothrin (172), cyfluthrin (176), (beta)-cyfluthrin (177), (lambda)-cyhalothrin (180), cypermethrin (183), (alpha)cypermethrin (184), (beta)-cypermethrin (185), (zeta)-cypermethrin (187), deltamethrin (204), esfenvalerate (277), fenpropathrin (312), fenvalerate (319), flucythrinate (333), (tau)-fluvalinate (362), permethrin (561), tefluthrin (687), tralomethrin (718) and ZXI 8901 (759);

c) insecticidal carbamates selected from the group consisting of:

alanycarb (15), aldicarb (16), amitraz (22), bendiocarb (56), benfuracarb (58), butocarboxim (95), carbaryl (106), carbofuran (109), carbosulfan (110), ethiofencarb (282), formetanate (369), furathiocarb (376), isoprocarb (431), methiocarb (482), methomyl (483), oxamyl (541), pirimicarb (583), propoxur (610), thiofanox (709), thiodicarb (708) and trimethacarb (743);

d) biopesticides selected from the group consisting of:

Bacillus thuringiensis (46, 47), Bacillus firmus, granulosis and nuclear polyhedrosis viruses, Beauveria bassiana (52), Beauveria brogniartii (53) and baculoviruses, such as Autographa california;

e) insecticidal growth regulators selected from the group consisting of: chlorfluazuron (125), DBI-3204, diflubenzuron (231), flucycloxuron (332), flufenoxuron (335), hexaflumuron (399), lufenuron (446), novaluron (527), methoxyfenozide (643), teflubenzuron (686), tebufenozide (679) and triflumuron (739);

f) others:

abamectin (1), bensultap (64), cartap (113), chlordane (121), chlorfenapyr (123), DNOC (261), endosulfan (270), fipronil (323), ethiprole, imidacloprid (418), thiacloprid, phosphine (574), oleic acid/fatty acids (532), pymetrozine (615), thiocyclam (707), IKI-220, tolfenpyrad (NEW), acetamiprid (5), spinosad (754), silafluofen (650) and thiamethoxam (NEW).

The numbers in brackets are the entry number in ‘The e-Pesticide Manual’, CD-ROM-Version 1.1, 1999-2000 (ISBN: 1-901396-22-3), based on The Pesticide Manual, 11th Edition, British Crop Protection Council, Farnham, 1997.

Baculoviruses are described, for example, in J. Ind. Microbiol. & Biotech 1997, 19, 192. Bacillus firmus, DBI-3204, Thiacloprid and IKI-220 are described in, for example, Proceedings of the BCPC-Conference, Pest & Diseases, 2000.

These references and the literature cited therein are herewith expressly referred to; they are incorporated into the description by reference.

The inventive method makes possible a reduced application rate of crop protection products which act synergistically with the transgenic plants, in addition to an increased and wider-ranging efficiency of said transgenic plants, thus offering economic and ecological advantages.

The advantages of the inventive method are firstly synergisms with the Bacillus thuringiensis toxins (Bt toxins) which are produced in the transgenic plant and secondly, for example, a reduced number of applications or reduced application rates to in some cases sublethal dosages (in comparison with the conventional use of the individual insecticides), and the markedly reduced pollution of the environment which this entails.

In particular, combinations of the abovementioned active substances, together with the endogenous Bt toxins, i.e. the Bt toxins produced within the transgenic plant, have a pronounced synergistic effect on a multiplicity of harmful organisms to be controlled.

Likewise, the invention relates to the use of compounds from among the abovementioned groups (a) to (f) for controlling harmful organisms in genetically modified vegetable plants which contain a gene derived from Bacillus thuringiensis which encodes, and expresses, an insecticidally active protein.

For the purposes of the invention, the term “insecticidally active” encompasses an insecticidal, acaricidal, molluscicidal, nematicidal, ovicidal effect and a repellant, behavior-modifying and sterilant effect.

Preferred insecticidal active substances are the abovementioned insecticidal organophosphorus compounds, pyrethroids, insecticidal carbamates, insecticidal growth regulators, abamectin, acetamiprid, chlorfenapyr, endosulfan, fipronil, ethiprole, imidacloprid, pymetrozine, spinosad, silafluofen, thiamethoxam, thiacloprid, tolfenpyrad, IKI-220 and Bacillus thuringiensis.

Especially preferred are ethoprophos, malathion, parathion, phosalone, pirimiphosmethyl, triazophos, acrinathrin, deltamethrin, tefluthrin, aldicarb, amitraz, bendiocarb, carbaryl, carbofuran, oxamyl, pirimicarb, thiodicarb, diflubenzuron, lufenuron, novaluron, methoxyfenozide, tebufenozide, abamectin, acetamiprid, chlorfenapyr, endosulfan, fipronil, ethiprole, imidacloprid, pymetrozine, spinosad, silafluofen, thiamethoxam, thiacloprid, tolfenpyrad, IKI-220 and Bacillus thuringiensis.

Very especially preferred are thiodicarb, acetamiprid, fipronil and tolfenpyrad.

Also preferred are mixtures of two or more, preferably two or three, particularly preferably two, of the insecticidally active compounds.

Particularly preferred are mixtures of the abovementioned organophosphorus compounds with the abovementioned pyrethroids.

Very particularly preferred are the mixtures listed hereinbelow: deltamethrin and endosulfan, deltamethrin and spinosad, deltamethrin and chlorphenapyr, deltamethrin and Bacillus thuringiensis, deltamethrin and methoxyfenozide, deltamethrin and tebufenozide, endosulfan and amitraz, endosulfan and Bacillus thuringiensis, cyfluthrin and chlorpyriphos.

Likewise preferred are mixtures of the abovementioned pyrethroids with imidacloprid, and mixtures of the abovementioned pyrethroids with tebufenozide.

The insecticidally active compounds employed in accordance with the invention are known; most of them are commercially available.

The insecticides used in accordance with the invention are usually obtainable as commercial formulations; however, they can be formulated in various ways, if appropriate, depending on the prevailing biological and/or chemico-physical parameters. The following are examples of possible formulations:

wettable powders (WP), emulsifiable concentrates (EC), aqueous solutions (SL), emulsions, sprayable solutions, oil- or water-based dispersions (SC), suspoemulsions (SE), dusts (DP), seed-dressing materials, granules in the form of microgranules, spray granules, coated granules and adsorption granules, water-dispersible granules (WG), ULV formulations, microcapsules, waxes or baits.

These individual formulation types are known in principle and are described, for example, in:

Winnacker-Kuichler, “Chemische Technologie” [Chemical Technology], Volume 7, C. Hauser Verlag Munich, 4th Ed. 1986; van Falkenberg, “Pesticides Formulations”, Marcel Dekker N.Y., 2nd Ed. 1972-73; Martens, “Spray Drying Handbook”, 3rd Ed. 1979, G. Goodwin Ltd. London.

The formulation auxiliaries required, such as inert materials, surfactants, solvents and further additives, are likewise known and are described, for example, in:

Watkins, “Handbook of Insecticide Dust Diluents and Carriers”, 2nd Ed., Darland Books, Caldwell N.J.; v. Olphen, “Introduction to Clay Colloid Chemistry”, 2nd Ed., J. Wiley & Sons, N.Y.; Marsden, “Solvents Guide”, 2nd Ed., Interscience, N.Y. 1950; McCutcheon's, “Detergents and Emulsifiers Annual”, MC Publ. Corp., Ridgewood N.J.; Sisley and Wood, “Encyclopedia of Surface Active Agents”, Chem. Publ. Co. Inc., N.Y. 1964; Schonfeldt, “Grenzflätchenaktive Äthylenoxidaddukte” [Interface-active ethylene oxide adducts], Wiss. Verlagsgesell., Stuttgart 1967; Winnacker-Küchler, “Chemische Technologie”, Volume 7, C. Hanser Verlag Munich, 4th Ed. 1986.

Combinations with other pesticidally active materials, fertilizers and/or plant growth regulators may also be prepared on the basis of these formulations, for example in the form of a readymix or a tankmix. Wettable powders are preparations which are uniformly dispersible in water and which, in addition to the active substance, also contain wetters, for example polyoxyethylated alkylphenols, polyoxyethylated fatty alcohols, alkylsulfonates or alkylphenolsulfonates and dispersants, for example sodium lignosulfonate, sodium 2,2′-dinaphthylmethane-6,6′-disulfonate, in addition to a diluent or inert substance.

Emulsifiable concentrates are prepared by dissolving the active substance in an organic solvent, for example butanol, cyclohexanone, dimethylformamide, xylene or else higher-boiling aromatics or hydrocarbons with addition of one or more emulsifiers. Emulsifiers which can be used are, for example, calcium alkylarylsulfonates such as calcium dodecylbenzenesulfonate, or nonionic emulsifiers such as fatty acid polyglycol esters, alkylaryl polyglycol ethers, fatty alcohol polyglycol ethers, propylene oxide/ethylene oxide condensates, alkyl polyethers, sorbitan fatty acid esters, polyoxyethylene sorbitan fatty acid esters or polyoxyethylene sorbitol esters.

Dusts are obtained by grinding the active substance with finely divided solid materials, for example talc, natural clays such as kaolin, bentonite and pyrophillite or diatomaceous earth. Granules can be prepared either by spraying adsorptive granulated inert material with the active substance or by applying active substance concentrates to the surface of carriers such as sand, kaolinites or granulated inert material with the aid of binders, for example polyvinyl alcohol, sodium polyacrylate or else mineral oils. Suitable active substances can also be granulated in the fashion which is customary for the production of fertilizer granules, if desired as a mixture with fertilizers.

The active substance concentration in wettable powders is, for example, approximately 10 to 90% by weight, the remainder to 100% by weight being composed of customary formulation constituents. In the case of emulsifiable concentrates, the active substance concentration can amount to approximately 5 to 80% by weight. Formulations in the form of dusts usually contain 5 to 20% by weight of active substance, while sprayable solutions contain approximately 2 to 20% by weight of active substance. In the case of granules, the active substance content depends partly on whether the active compound is in liquid or solid form and on the granulation auxiliaries, fillers and the like which are being used.

In addition, the abovementioned active substance formulations contain, if appropriate, the auxiliaries which are conventional in each case, such as stickers, wetters, dispersants, emulsifiers, penetrants, solvents, fillers or carriers.

For use, the concentrates, which are present in commercially available form, are, if appropriate, diluted in the customary fashion, for example using water in the case of wettable powders, emulsifiable concentrates, dispersions and in some cases also in the case of microgranules. Preparations in the form of dusts, granulated preparations and sprayable solutions are usually not diluted any further with other inert substances prior to use.

The application rate required varies depending on the external conditions such as temperature, humidity and the like. It can vary within wide limits, for example between 0.1 g/ha and 5.0 kg/ha or more of active substance. However, it is preferably between 0.1 g/ha and 1.0 kg/ha. Owing to the synergistic effects between Bt vegetables and insecticide, application rates of from 0.1 to 500 g/ha are particularly preferred.

In the case of insecticidal organophosphorus compounds (a), preferred application rates are 50 to 500 g/ha, particularly preferred application rates are 50 to 200 g/ha.

In the case of pyrethroids (b), preferred application rates are 0.1 to 10 g/ha, particularly preferred are 0.1 to 6.0 g/ha.

In the case of insecticidal carbamates (c), preferred application rates are 50 to 5 000 g/ha, particularly preferred are 50 to 2 000 g/ha.

In the case of biopesticides (d), the commercially customary application rates are preferred.

In the case of insecticidal growth regulators (e), application rates of 10 to 1 000 g/ha are preferred, particularly preferred are 50 to 500 g/ha.

In the case of the insecticides of group (f), application rates of 0.1 to 5 000 g/ha are preferred, 0.1 to 300 g/ha are particularly preferred.

The active substances according to the invention, in their commercially available formulations and in the use forms prepared from these formulations, may be present in the form of mixtures with other active substances, such as insecticides, attractants, sterilants, acaricides, nematicides, fungicides, growth-regulatory substances or herbicides.

Preferred other components in the mixtures are

1. from the group of the carboxylic esters,

alphametrin, 5-benzyl-3-furylmethyl-(E)-, (1R)-cis-2,2-dimethyl-3-(2-oxothiolan-3-yl-idenemethyl)cyclopropanecarboxylate, bioallethrin, bioallethrin ((S)-cyclopentyl isomer), bioresmethrin, (RS)-1-cyano-1-(6-phenoxy-2-pyridyl)methyl (1RS)-trans-3-(4-tert-butylphenyl)-2,2-dimethylcyclopropanecarboxylate (NCI 85193), cythithrin, cyphenothrin, empenthrin, fenfluthrin, flumethrin, fluvalinate (D isomer), imiprothrin (S-41311), phenothrin ((R) isomer), prallethrin, pyrethrine (natural products), resmethrin, tetramethrin, theta-cypermethrin (TD-2344), transfluthrin;

2. from the group of the amidines,

chlordimeform;

3. others:

ABG-9008, Anagrapha falcitera, AKD-1022, AKD-3059, bifenazate (D-2341), binapacryl, BJL-932, bromopropylate, BTG-504, BTG-505, buprofezin, camphechlor, chlorobenzilate, 2-(4-chlorophenyl)4,5-diphenylthiophene (UBI-T 930), chlorfentezine, chromafenozide (ANS-118), CG-216, CG-217, CG-234, A-184699, 2-naphthylmethyl cyclopropanecarboxylate (Ro12-0470), cyromazin, diacloden, ethyl N-(3,5-dichloro-4-(1,1,2,3,3,3-hexafluoro-1-propyloxy)phenyl)carbamoyl)-2-chlorobenzocarboximidate, dicofol, N-(2,3-dihydro-3-methyl-1,3-thiazol-2-ylidene)-2,4-xylidine, dinobuton, dinocap, diofenolan, DPX-062, emamectin benzoate (MK-244), sulfethiprole, ethofenprox, etoxazole (YI-5301), fenoxycarb, fluazuron, flumite (flufenzine, SZI-121), 2-fluoro-5-(4-(4-ethoxyphenyl)-4-methyl-1-pentyl)diphenyl ether (MTI 800), fenpyroximate, fenthiocarb, flubenzimine, flufenprox (ICI-A5683), fluproxyfen, gamma-HCH, halofenozide (RH-0345), halofenprox (MTI-732), hexythiazox, HOI-9004, hydramethyinon (AC 217300), indoxacarb (DPX-MP062), kanemite (AKD-2023), M-020, MTI446, ivermectin, M-020, milbemectin, NC-196, Neemgard, nitenpyram (TI-304), 2-nitromethyl-4,5-dihydro-6H-thiazine (DS 52618), 2-nitromethyl-3,4-dihydrothiazole (SD 35651), 2-nitromethylene-1,2-thiazinan-3-ylcarbamaldehyde (WL 108477), pyriproxyfen (S-71639), NC-196, NC-1111, NNI-9768, OK-9701, OK-9601, OK-9602, propargite, pyridaben, pyrimidifen (SU-8801), RH-0345, RYI-210, S-1283, S-1833, SB7242, SI-8601, silomadine (CG-177), SU-9118, tebufenpyrad (MK-239), tetradifon, tetrasul, TI-435, verbutin, vertalec (Mykotal), YI-5301.

The active substance content of the use forms prepared from the commercially available formulations may range from 0.00000001 up to 95% by weight and is preferably between 0.00001 and 1% by weight of active substance.

For example, corresponding formulations of mixtures of pyrethroids and organophosphorus compounds contain 0.05 to 0.01% by weight of pyrethroid and 0.25 to 0.20% by weight of organophosphorus compound, preferably 0.01 to 0.001% by weight of pyrethroid and 0.2 to 0.1% by weight of organophosphorus compound.

In the case of mixtures of pyrethroids and endosulfan, a ratio of 0.05 to 0.01% by weight of pyrethroid to 0.7 to 0.2% by weight of endosulfan is preferred, particularly preferred are 0.01 to 0.001% by weight of pyrethroid and 0.35 to 0.2% by weight of endosulfan.

In the case of mixtures of pyrethroids and Bacillus thuringiensis (Bt), the data given above for pyrethroids apply, while the Bt fraction preferably amounts to 0.01 to 0.001% by weight, particularly preferably 0.005 to 0.001% by weight.

Mixtures of endosulfan and amitraz preferably contain 0.35 to 0.2% by weight of endosulfan and 0.6 to 0.2% by weight of amitraz.

The inventive method is preferably suitable for use against all developmental stages of the harmful organisms (egg, all instars such as, for example, L1, L2, L3, L4, L5 to adult), in particular in the control of Homoptera, Diptera, Lepidoptera and Coleoptera.

For the purposes of the invention, the term “Bt vegetable” is understood as referring to vegetable plants or vegetable crops which are genetically modified and contain, one or more Bacillus thuringiensis genes which in turn encode and express, insecticidally active proteins. These plants (“Bt vegetables”) can be grown both in the open and under the conditions found when growing crops under glass. These vegetable plants or crops include, for example,

potatoes, preferably starch potatoes, sweet potatoes and table potatoes;

root vegetables, preferably

carrots, rutabaga (table beet, stubble turnips, turnips, Brassica rapa. var. rapa f. teltowiensis), scorzoneras, Jerusalem artichoke, root parsley, parsnip, radish and horseradish;

tuberous vegetables, preferably

kohlrabi, red beet, celeriac, radish;

bulbous vegetables, preferably

leeks and onions (onion sets and onions for seed production);

cabbages, preferably

cabbages from the Capitata group (white cabbage, red cabbage, kale, savoy cabbage), cauliflower, Brussels sprouts, broccoli, Brassica oleracea. var. sabellica, stem kale, seakale and Brassica oleracea L. convar. oleracea var. gemmifera DC.;

fruiting vegetables, preferably

tomatoes (field-grown tomatoes, bush tomatoes, beefsteak tomatoes, greenhouse-grown tomatoes, cocktail tomatoes, processing tomatoes and tomatoes to be sold fresh), melons, egg plants, aubergines, capsicums (bell peppers, paprika, Spanish pepper), chillis, pumpkins, zucchini and cucumbers (field-grown cucumbers, greenhouse-grown cucumbers, snake cucumbers, gherkins);

vegetable legumes, preferably

dwarf beans (as sword beans, beech beans, flageolet beans, butter beans; dried beans for boiling with green- and yellow-podded varieties), pole beans (as sword beans, beech beans, flageolet beans, butter beans with green-, blue- and yellow-podded varieties), faba beans (field beans, broad beans, varieties with white and black mottled flowers), peas (chickling vetches, chick peas, marrowfat peas, whole-pod peas, sugar peas, peas for shelling, varieties with light-green and dark-green immature seeds) and lentils;

leaf and stem vegetables, preferably

Chinese cabbage, lettuce, cos lettuce, corn salad, iceberg lettuce, romaine lettuce, oak-leaf lettuce, chicory, radicchio, lollo rosso, arugula, endives, spinach, Swiss chard (leaves and stems) and parsley;

other vegetables, preferably

asparagus, rhubarb, chives, artichokes, mints, sunflowers, Florence fennel, dillweed, garden cress, mustard, poppies, peanuts, sesame and chicories for salad use.

All those plants which come under the generic term “Bt vegetables” are, for the purposes of the invention, genetically modified in such a way that they contain, and express, one or more genes from Bacillus thuringiensis which encode an insecticidally active protein.

Preferred are Bacillus thuringiensis crystal proteins from the Cry family (see, for example, Crickmore et al., Microbiol. Mol. Biol. Rev. 1998, 62, 807-812), which are active against Lepidoptera, Coleoptera and Diptera.

Particularly preferred are genes encoding the proteins cry1Aa1, cry1Aa2, cry1Aa3, cry1Aa4, cry1Aa5, cry1Aa6, cry1Aa7, cry1Aa8, cry1Aa9, cry1Aa10, cry1Aa11 cry1Ab1, cry1Ab2, cry1Ab3, cry1Ab4, cry1Ab5, cry1Ab6, cry1Ab7, cry1Ab8, cry1Ab9, cry1Ab10, cry1Ab11, cry1Ab12, cry1Ab13, cry1Ab14, cry1Ac1, cry1Ac2, cry1Ac3, cry1Ac4, cry1Ac5, cry1Ac6, cry1Ac7, cry1Ac8, cry1Ac9, cry1Ac10, cry1Ac11, cry1Ac12, cry1Ac13, cry1Ad1, cry1Ad2, cry1Ae1, cry1Af1, cry1Ag1, cry1Ba₁, cry1Ba2, cry1Bb1, cry1Bc1, cry1Bd1, cry1Be1, cry1Ca11, cry1Ca2, cry1Ca3, cry1Ca4, cry1Ca5, cry1Ca6, cry1Ca7, cry1Cb11, cry1Cb2, cry1Da1, cry1Da2, cry1Db1, cry1Ea1, cry1Ea2, cry1Ea3, cry1Ea4, cry1Ea5, cry1Ea6, cry1Eb1, cry1Fa1, cry1Fa2, cry1Fb1, cry1Fb2, cry1Fb3, cry1F1Fb4, cry1Ga1, cry1Ga2, cry1Gb1, cry1Gb2, cry1Ha1, cry1Hb1, cry1Ia1, cry1Ia2, cry1Ia3, cry1Ia4, cry1a5, cry1Ia6, cry1Ib1, cry1Ic1, cry1Id1, cry1Ie1, cry1I-like, cry1Ja1, cry1Jb1, cry1Jc1, cry1Ka1, cry1-like, cry2Aa1, cry2Aa2, cry2Aa3, cry2Aa4, cry2Aa5, cry2Aa6, cry2Aa7, cry2Aa8, cry2Aa9, cry2Ab1, cry2Ab2, cry2Ab3, cry2Ac1, cry2Ac2, cry2Ad1, cry3Aa1, cry3Aa2, cry3Aa3, cry3Aa4, cry3Aa5, cry3Aa6, cry3Aa7, cry3Ba1, cry3Ba2, cry3Bb1, cry3Bb2, cry3Bb3, cry3Ca1, cry4Aa1, cry4Aa2, cry4Ba1, cry4Ba2, cry4Ba3, cry4Ba4, cry5Aa1, cry5Ab1, cry5Ac1, cry5Ba1, cry6Aa1, cry6Ba1, cry7Aa1, cry7Ab1, cry7Ab2, cry8Aa1, cry8Ba1, cry8Ca1, cry9Aa1, cry9Aa2, cry9Ba1, cry9Ca1, cry9Da1, cry9Da2, cry9Ea1, cry9 like, cry10Aa1, cry10Aa2, cry11Aa1, cry11Aa2, cry11Ba1, cry11Bb1, cry12Aa1, cry13Aa1, cry14Aa1, cry15Aa1, cry16Aa1, cry17Aa1, cry18Aa1, cry18Ba1, cry18Ca1, cry19Aa1, cry19Ba1, cry20Aa1, cry21Aa1, cry21Aa2, cry22Aa1, cry23Aa1, cry24Aa1, cry25Aa1, cry26Aa1, cry27Aa1, cry28Aa1, cry28Aa2, cry29Aa1, cry30Aa1, cry31Aa1, cyt1Aa1, cyt1Aa2, cyt1Aa3, cyt1Aa4, cyt1Ab1, cyt1Ba1, cyt2Aa1, cyt2Ba1, cyt2Ba2, cyt2Ba3, cyt2Ba4, cyt2Ba5, cyt2Ba6, cyt2Ba7, cyt2Ba8, cyt2Bb1.

Particularly preferred are the subfamilies cry1, cry2, cry3, cry5 and cry9.

Very particularly preferred are cry1Ab, cry1Ac, cry3A, cry3B and cry9C.

Moreover, it is preferred to employ plants which contain genes for more than one Bt protein.

In addition to the expression of Bacillus thuringiensis (Bt) toxins for insect resistance, the transgenic crop plants may also have further transgenic characteristics, for example further insect resistances (for example owing to the expression of a protease or peptidase inhibitor, cf. WO-A-95/35031), herbicide resistances (for example against glufosinate or glyphosate owing to expression of the pat or bar gene) or else resistances to nematodes, fungi or viruses (for example owing to the expression of a glucanase, chitinase), or else be genetically modified in their metabolic characteristics so that a qualitative and/or quantitative modification of constituents results (for example owing to modification of the energy, carbohydrate, fatty acid or nitrogen metabolism or metabolite fluxes which influence them). Examples of preferred Bt vegetable plants are those which additionally have a glufosinate or glyphosate resistance.

Bt vegetable plants, including methods for their generation, are described extensively in, for example, Barton et al., 1987, Plant Physiol. 85: 1103-1109; Vaeck et al., 1987, Nature 328:33-37; Fischhoff et al., 1987, Bio/Technology 5: 807-813.

Bt vegetable plants are furthermore obtainable commercially in various variations, for example the potato variety NewLeaf® from Monsanto. Further vegetable varieties are being developed and/or trialled, such as, for example, a tomato variety with resistance to lepidopterens, by Monsanto.

Ways of generating transgenic plants which, in comparison with naturally occurring plants, have modified characteristics consist for example in the use of the methods mentioned in the text which follows: Willmitzer, 1993, Transgenic plants, in: Biotechnology, A Multivolume Comprehensive Treatise, Rehm et al. (eds.) Vol.2, 627-659, VCH Weinheim, Germany; McCormick et al. Plant Cell Reports,1986, 5, 81-84, EP-A-0221044, EP-A-0131624.

A large number of molecular biology techniques by means of which novel transgenic plants with modified characteristics can be generated are known to the skilled worker; see, for example, Sambrook et al., 1989, Molecular Cloning, A Laboratory Manual, 3rd Ed., Cold Spring Harbor Laboratory Press, Cold Spring Harbor, N.Y.; Winnacker “Gene und Klone” [Genes and clones], VCH Weinheim, 2nd Ed. 1996, or Christou, Trends in Plant Science 1 (1996) 423-431.

To carry out such recombinant manipulation, suitable nucleic acid molecules can be introduced into plants or plant cells, for example by means of suitable vectors which allow mutagenesis or a change in the sequence to take place by the recombination of DNA sequences. With the aid of the abovementioned standard methods, it is also possible, for example, to carry out base substitutions, to remove part sequences or to add natural or synthetic sequences. Also, it is possible, for example, to replace the naturally occurring genes completely by heterologous or synthetic genes, preferably under the control of a promoter which is active in plant cells (gene replacement). To link the DNA fragments with one another, the fragments can be provided with adapters or linkers.

Plant cells with a reduced activity of a gene product can be obtained, for example, by expressing at least one corresponding antisense RNA, a sense RNA for achieving a cosuppression effect, or expressing at least one suitably constructed ribozyme which specifically cleaves transcripts of the abovementioned gene product.

To this end, it is possible, on the one hand, to use DNA molecules which encompass all of the coding sequence of a gene product including any flanking sequences which may be present, but also DNA molecules which only encompass portions of the coding sequence, it being necessary for these portions to be so long as to cause an antisense effect in the cells. Another possibility is the use of DNA sequences which have a high degree of homology with the coding sequences of a gene product, but are not completely identical.

When expressing nucleic acid molecules in plants, the protein synthesized may be localized in any desired compartment of the plant cell. However, to achieve localization in a particular compartment, the coding region can, for example, be linked to DNA sequences which ensure localization in a particular compartment or at a particular point in time (induced at a particular stage, or chemically or biologically induced; for example transit or signal peptides, timing- or location-specific promoters). Such sequences are known to the skilled worker (see, for example, Braun et al., EMBO J. 11 (1992), 3219-3227; Wolter et al., Proc. Natl. Acad. Sci. USA 85 (1988), 846-850; Sonnewald et al., Plant J. 1 (1991), 95-106).

The transgenic plant cells can be regenerated by known techniques to give intact plants.

In this manner, transgenic plants can be obtained which exhibit modified characteristics owing to the overexpression, suppression or inhibition of homologous (i.e. endogenous) genes or gene sequences or the expression of heterologous (i.e. exogenous) genes or gene sequences.

The inventive method is suitable for controlling a multiplicity of harmful organisms which are found in particular in vegetables, in particular insects and arachnids, very particularly preferably insects. The abovementioned pests include:

from the order of the Isopoda, for example, Armadillidium spp., Oniscus spp., Porcellio spp..

from the order of the Diplopoda, for example, Blaniulus spp..

from the order of the Chilopoda, for example, Geophilus spp., Scutigera spp..

from the order of the Symphyla, for example, Scutigerella spp..

from the order of the Thysanura, for example, Lepisma spp..

from the order of the Collembola, for example, Onychiurus spp..

from the order of the Orthoptera, for example, Blattella spp., Periplaneta spp., Leucophaea spp., Acheta spp., Gryllotalpa spp., Locusta migratoria migratorioides, Melanoplus spp., Schistocerca spp..

from the order of the Dermaptera, for example, Forficula spp..

from the order of the Isoptera, for example, Reticulitermes spp., Reticulitermes spp., Coptotermes spp..

from the order of the Thysanoptera, for example, Frankliniella spp., Kakothrips spp., Hercinothrips spp., Scirtothrips spp., Taeniothrips spp., Thrips spp..

from the order of the Heteroptera, for example, Eurygaster spp., Stephanitis spp., Lygus spp., Aelia spp., Eurydema spp., Dysdercus spp., Piesma spp..

from the order of the Homoptera, for example, Aleurodes spp., Bemisia spp., Trialeurodes spp., Brevicoryne spp., Cryptomyzus spp., Aphis spp., Eriosoma spp., Hyalopterus spp., Phylloxera spp., Pemphigus spp., Macrosiphum spp., Myzus spp., Phorodon spp., Rhopalosiphum spp., Empoasca spp., Euscelis spp., Eulecanium spp., Saissetia spp., Aonidiella spp., Aspidiotus spp., Nephotettix spp., Laodelphax spp., Nilaparvata spp., Sogatella spp., Pseudococcus spp., Psylla spp., Aphrophora spp., Aeneolamia spp.

from the order of the Lepidoptera, for example, Pectinophora spp., Bupalus spp., Cheimatobia spp., Cnephasia spp., Hydraecia spp., Lithocolletis spp., Hyponomeuta spp., Plutella spp., Plutella xylostella, Malacosoma spp., Euproctis spp., Lymantria spp., Bucculatrix spp., Phytometra spp., Scrobipalpa spp., Phthorimaea spp., Gnorimoschema spp., Autographa spp., Evergestis spp., Lacanobia spp., Cydia spp., Pseudociaphila spp., Phyllocnistis spp., Agrotis spp., Euxoa spp., Feltia spp., Earias spp., Heliothis spp., Helicoverpa spp., Bombyx spp., Laphygma spp., Mamestra spp., Panolis spp., Prodenia spp., Spodoptera spp., Trichoplusia spp., Carpocapsa spp., Pieris spp., Chilo spp., Ostrinia spp., Pyrausta spp., Ephestia spp., Galleria spp., Cacoecia spp., Capua spp., Choristoneura spp., Clysia spp., Hofmannophila spp., Homona spp., Tineola spp., Tinea spp., Tortrix spp..

from the order of the Coleoptera, for example, Anobium spp., Rhizopertha spp., Bruchidius spp., Acanthoscelides spp., Hylotrupes spp., Aclypea spp., Agelastica spp., Leptinotarsa spp., Psylliodes spp., Chaetocnema spp., Cassida spp., Bothynoderes spp., Clivina spp., Ceutorhynchus spp., Phyllotreta spp., Apion spp., Sitona spp., Bruchus spp., Phaedon spp., Diabrotica spp., Psylloides spp., Epilachna spp., Atomaria spp., Oryzaephilus spp., Anthonomus spp., Sitophilus spp., Otiorhynchus spp., Cosmopolites spp., Ceuthorrynchus spp., Hypera spp., Dermestes spp., Trogoderma spp., Anthrenus spp., Attagenus spp., Lyctus spp., Meligethes spp., Ptinus spp., Niptus spp., Gibbium spp., Tribolium spp., Tenebrio spp., Agriotes spp., Conoderus spp., Melolontha spp., Amphimallon spp., Costelytra spp..

from the order of the Hymenoptera, for example, Diprion spp., Hoplocampa spp., Lasius spp., Monomorium spp., Vespa spp..

from the order of the Diptera, for example, Drosophila spp., Chrysomyxa spp., Hypoderma spp., Tannia spp., Bibio spp., Oscinella spp., Phorbia spp., Pegomyia spp., Ceratitis spp., Dacus spp., Tipula spp..

from the class of the Arachnida, for example, Scorpio maurus, Latrodectus mactans.

from the order of the Acarina, for example, Acarus spp., Bryobia spp., Panonychus spp., Tetranychus spp., Eotetranychus spp., Oligonychus spp., Eutetranychus spp., Eriophyes spp., Phyllocoptruta spp., Tarsonemus spp., Rhipicephalus spp., Rhipicephalus spp., Ixodes spp., Amblyomma spp..

from the class of the Helminthen, for example, Meloidogyne spp., Heterodera spp., Globodera spp., Radopholus spp., Pratylenchus spp., Tylenchulus spp., Tylenchorhynchus spp., Rotylenchus spp., Heliocotylenchus spp., Belonoaimus spp., Longidorus spp., Trichodorus spp., Xiphinema spp., Ditylenchus spp., Aphelenchoides spp., Anguina spp..

from the class of the Gastropoda, for example, Deroceras spp., Arion spp., Lymnaea spp., Galba spp., Succinea spp..

The inventive method is preferably suitable for controlling Agriotes spp., Melolontha spp., Aphis spp., Cnephasia spp., Ostrinia spp., Agrotis spp., Hydraecia spp., Tipula spp., Myzus spp., Bemisia spp., Trialeurodes spp., Oscinella spp., Tetranychus spp., Lygus spp., Leptinotarsa spp., Psylliodes spp., Phytometra spp., Deroceras spp., Psylla spp., Blaniulus spp., Onychiurus spp., Piesma spp., Atomaria spp., Aclypea spp., Chaetocnema spp., Cassida spp., Bothynoderes spp., Clivina spp., Scrobipalpa spp., Phthorimaea spp., Gnorimoschema spp., Mamestra spp., Autographa spp., Arion spp., Gryllotalpa spp., Eurydema spp., Meligethes spp., Ceutorhynchus spp., Phyllotreta spp., Plutella xylostella, Evergestis spp., Lacanobia spp., Pieris spp., Forficula spp., Hypera spp., Apion spp., Otiorhynchus spp., Sitona spp., Acanthoscelides spp., Kakothrips spp., Bruchus spp., Cydia spp., Thrips spp., Pseudociaphila spp., Heliothis spp., Helicoverpa spp., Prodenia spp., Spodoptera spp., Chilo spp and Diabrotica spp..

The invention is now illustrated in greater detail by the examples which follow, without being limited thereto.

### EXAMPLE

### Plutella xylostella (Diamondback Moth)

Seven-week-old cauliflower plants (ordinary cauliflower, variety: Marner Allfrüh) and Bt cauliflower (Bt-resistant strain, contains cry9C) were sprayed with the test insecticides with the aid of a track sprayer (200 l/ha). After drying, each of the plants was infected with 5 L2 larvae and 5 L3 larvae of Plutella xylostella. After 2, 4 and 7 days (DM), feeding damage and mortality were scored. The test was carried out in a greenhouse at 23° C. and a relative atmospheric humidity of 60%.

| | | % feeding damage/% mortality | | | | | | | | | | | |
|---|---|---|---|---|---|---|---|---|---|---|---|---|---|
| Compound | | 2 DAA | | | | 4 DAA | | | | 7 DAA | | | |
| g active | | Cauli- | | Bt. | | Cauli- | | Bt. | | Cauli- | | Bt. | |
| substance/ha | | flower | | cauliflower | | flower | | cauliflower | | flower | | cauliflower | |
| Control | — | 10 | 20 | 10 | 0 | 15 | 20 | 15 | 20 | 15 | 20 | 15 | 30 |
| Tolfenpyrad | 100 | 5 | 0 | 10 | 20 | 5 | 20 | 10 | 100 | 5 | 0 | 10 | 100 |
| EC15 | 30 | 10 | 0 | 0 | 60 | 10 | 0 | 0 | 100 | 10 | 0 | 0 | 100 |
| | 10 | 10 | 0 | 10 | 0 | 10 | 0 | 10 | 50 | 10 | 0 | 10 | 50 |
| | 3 | 10 | 0 | 3 | 10 | 10 | 0 | 10 | 30 | 10 | 0 | 10 | 50 |
| Fipronil | 100 | 1 | 100 | 0 | 100 | 1 | 100 | 0 | 100 | 1 | 100 | 0 | 100 |
| SC05 | 30 | 4 | 70 | 0 | 100 | 5 | 100 | 0 | 100 | 5 | 100 | 0 | 100 |
| | 10 | 8 | 20 | 0 | 100 | 8 | 20 | 0 | 100 | 10 | 50 | 0 | 100 |
| | 3 | 10 | 20 | 0 | 100 | 10 | 20 | 0 | 100 | 12 | 20 | 0 | 100 |
| | 1 | 10 | 0 | 0 | 70 | 10 | 0 | 0 | 100 | 12 | 0 | 0 | 100 |
| Thiodicarb | 100 | 10 | 0 | 0 | 100 | 10 | 0 | 0 | 100 | 10 | 0 | 0 | 100 |
| SC33 | 30 | 10 | 0 | 0 | 100 | 10 | 0 | 0 | 100 | 15 | 0 | 0 | 100 |
| | 10 | 10 | 0 | 0 | 90 | 10 | 0 | 0 | 100 | 15 | 0 | 0 | 100 |
| | 3 | 10 | 0 | 0 | 100 | 10 | 0 | 0 | 100 | 15 | 0 | 0 | 100 |
| | 1 | 10 | 0 | 0 | 100 | 10 | 0 | 0 | 100 | 15 | 0 | 0 | 100 |
| Acetamiprid | 30 | 10 | 0 | 0 | 100 | 12 | 0 | 0 | 100 | 15 | 0 | 0 | 100 |
| WP20 | 10 | 10 | 0 | 0 | 100 | 12 | 0 | 0 | 100 | 15 | 0 | 0 | 100 |

A significant synergistic effect is observed clearly for all four active substances.

What is claimed is:

1. A method for controlling harmful organisms in genetically modified vegetable plants containing a gene derived from Bacillus thuringiensis, said gene encoding and expressing a protein with an insecticidal action, wherein an insecticidally active quantity of one or more compounds from the following groups (a) to (f) is applied to the plants, their seeds or propagation material and/or the area in which they are cultivated:
a) insecticidal organophosphorus compounds selected from the group consisting of:
acephate, azinphos-ethyl, azinphos-methyl, cadusafos, chlorfenvinphos, chlormephos, chlorpyrifos, demeton-S-methyl, diazinon, dicrotophos, dimethoate, disulfoton, ethion, ethoprophos, etrimfos, fonofos, isazofos, isofenphos, malathion, methamidophos, methidathion, mevinphos, monocrotophos, omethoate, parathion, phenthoate, phorate, phosalone, phosmet, phosphamidon, phoxim, pirimiphosmethyl, profenofos, prothiofos, pyridaphenthion, quinalphos, terbufos, tetrachlorvinphos and triazophos;
b) pyrethroids selected from the group consisting of:
acrinathrin, allethrin, bifenthrin, cycloprothrin, cyfluthrin, (beta)-cyfluthrin, (lambda)-cyhalothrin, cypermethrin, (alpha)-cypermethrin, (beta)-cypermethrin, (zeta)-cypermethrin, deltamethrin, esfenvalerate, fenpropathrin, fenvalerate, flucythrinate, (tau)-fluvalinate, permethrin, tefluthrin, tralomethrin and ZXl 8901;
c) insecticidal carbamates selected from the group consisting of:
alanycarb, aldicarb, amitraz, bendiocarb, benfuracarb, butocarboxim, carbaryl, carbofuran, carbosulfan, ethiofencarb, formetanate, furathiocarb, isoprocarb, methiocarb, methomyl, oxamyl, pirimicarb, propoxur, thiofanox, thiodicarb and trimethacarb;
d) biopesticides selected from the group consisting of:
Bacillus thuringiensis, Bacillus firmus, granulosis and nuclear polyhedrosis viruses, Beauveria bassiana, Beauveria brogniartii and baculoviruses, such as Autographa california;
e) insecticidal growth regulators selected from the group consisting of:
chlorfluazuron, DBI-3204, diflubenzuron, flucycloxuron, flufenoxuron, hexaflumuron, lufenuron, novaluron, methoxyfenozide, teflubenzuron, tebufenozide and triflumuron;
f) others:
abamectin, bensultap, cartap, chlordane, chlorfenapyr, DNOC, endosulfan, fipronil, ethiprole, imidacloprid, thiacloprid, phosphine, oleic acid/fatty acids, pymetrozine, thiocyclam, IKI-220, tolfenpyrad, acetamiprid, spinosad, silafluofen and thiamethoxam.

2. The method as claimed in claim 1, wherein a compound selected from the group of the insecticidal organophosphorus compounds (a), pyrethroids (b), insecticidal carbamates (c), insecticidal growth regulators (e), abamectin, acetamiprid, chlorfenapyr, endosulfan, fipronil, ethiprole, imidacloprid, pymetrozine, spinosad, silafluofen, thiamethoxam, thiacloprid, tolfenpyrad, IKI-220 and Bacillus thuringiensis is used.

3. The method as claimed in claim 2, wherein a compound selected from the group consisting of ethoprophos, malathion, parathion, phosalone, pirimiphosmethyl, triazophos, acrinathrin, deltamethrin, tefluthrin, aldicarb, amitraz, bendiocarb, carbaryl, carbofuran, oxamyl, pirimicarb, thiodicarb, diflubenzuron, lufenuron, novaluron, methoxyfenozide, tebufenozide, abamectin, acetamiprid, chlorfenapyr, endosulfan, fipronil, ethiprole, imidacloprid, pymetrozine, spinosad, silafluofen, thiamethoxam, thiacloprid, tolfenpyrad, IKI-220 and Bacillus thuringiensis is used.

4. The method as claimed in one or more of claims 1 to 3, wherein a mixture of two or more of the insecticidally active compounds is employed.

5. The method as claimed in one or more of claims 1 to 4, wherein the insecticidally active compound is applied at an application rate of from 0.0001 to 5.0 kg/ha.

6. The method as claimed in one or more of claims 1 to 5, wherein the insecticidally active compound is employed as a 0.00001 to 95% by weight formulation.

7. The method as claimed in one or more of claims 1 to 6, wherein the insecticidally active protein in the vegetable plant is a crystal protein from the subfamily cry1, cry2, cry3, cry5 and/or cry9.

8. The method as claimed in claim 7, wherein the insecticidally active protein in the vegetable plant is cry1Ab, cry1Ac, cry3A, cry3B and/or cry9C.

9. The method as claimed in one or more of claims 1 to 8, wherein the vegetable plants used have a glufosinate or glyphosate resistance.

10. The method as claimed in one or more of claims 1 to 9, wherein the harmful organisms are insects which belong to the orders Homoptera, Lepidoptera and/or Coleoptera.

11. The method as claimed in one or more of claims 1 to 10, wherein the insecticidally active compound is employed against eggs and/or against L1 and/or L2 and/or L3 and/or L4 and/or L5 instars and/or against adults of the harmful organisms.

12. The method as claimed in one or more of claims 1 to 11, wherein, in addition to one or more insecticidally active compounds selected from among groups (a) to (f), one or more further insecticidally, fungicidally or herbicidally active compound(s) is/are employed.

13. The use of compounds from the following groups (a) to (f)
a) insecticidal organophosphorus compounds selected from the group consisting of:
acephate, azinphos-ethyl, azinphos-methyl, cadusafos, chlorfenvinphos, chlormephos, chlorpyrifos, demeton-S-methyl, diazinon, dicrotophos, dimethoate, disulfoton, ethion, ethoprophos, etrimfos, fonofos, isazofos, isofenphos, malathion, methamidophos, methidathion, mevinphos, monocrotophos, omethoate, parathion, phenthoate, phorate, phosalone, phosmet, phosphamidon, phoxim, pirimiphosmethyl, profenofos, prothiofos, pyridaphenthion, quinalphos, terbufos, tetrachlorvinphos and triazophos;
b) pyrethroids selected from the group consisting of:
acrinathrin, allethrin, bifenthrin, cycloprothrin, cyfluthrin, (beta)-cyfluthrin, (lambda)-cyhalothrin, cypermethrin, (alpha)-cypermethrin, (beta)-cypermethrin, (zeta)-cypermethrin, deltamethrin, esfenvalerate, fenpropathrin, fenvalerate, flucythrinate, (tau)-fluvalinate, permethrin, tefluthrin, tralomethrin and ZXl 8901;
c) insecticidal carbamates selected from the group consisting of:
alanycarb, aldicarb, amitraz, bendiocarb, benfuracarb, butocarboxim, carbaryl, carbofuran, carbosulfan, ethiofencarb, formetanate, furathiocarb, isoprocarb, methiocarb, methomyl, oxamyl, pirimicarb, propoxur, thiofanox, thiodicarb and trimethacarb;
d) biopesticides selected from the group consisting of:
Bacillus thuringiensis, Bacillus firmus, granulosis and nuclear polyhedrosis viruses, Beauveria bassiana, Beauveria brogniartii and baculoviruses, such as Autographa california;
e) insecticidal growth regulators selected from the group consisting of:
chlorfluazuron, DBI-3204, diflubenzuron, flucycloxuron, flufenoxuron, hexaflumuron, lufenuron, novaluron, methoxyfenozide, teflubenzuron, tebufenozide and triflumuron;
f) others:
abamectin, bensultap, cartap, chlordane, chlorfenapyr, DNOC, endosulfan, fipronil, ethiprole, imidacloprid, thiacloprid, phosphine, oleic acid/fatty acids, pymetrozine, thiocyclam, IKI-220, tolfenpyrad, acetamiprid, spinosad, silafluofen and thiamethoxam;
for controlling harmful organisms in genetically modified vegetable plants containing a gene derived from Bacillus thuringiensis, said gene encoding and expressing an insecticidally active protein.
